(19) United States
(12) Patent Application Publication
(10) Pub. No.: US 2026/0192037 A1
(43) Pub. Date: Jul. 9, 2026

(54) MULTI-LUMEN DRAINAGE CONDUITS WITH PRE-EVACUATED LUMEN AND DRAINAGE LUMEN FOR DRAINING BODILY FLUIDS

(71) Applicant: Bard Peripheral Vascular, Inc., Franklin Lakes, NJ (US)

(72) Inventor: Wesley WALKER, Broken Arrow, OK (US)

(73) Assignee: Bard Peripheral Vascular, Inc., Franklin Lakes, NJ (US)

(21) Appl. No.: 18/863,849

(22) PCT Filed: Jun. 14, 2022
(86) PCT No.: PCT/US2022/033423

§ 371 Date: Nov. 7, 2024

(51) Int. Cl. A61M 1/00

(52) U.S. Cl. CPC A61M 1/913; A61M 1/70; A61M 1/96; A61M 2210/101

(57) ABSTRACT

A drainage system for draining bodily fluid includes a multi-lumen conduit comprising a drainage lumen configured to receive a bodily fluid therethrough and a pre-evacuated lumen that is pre-evacuated for providing a negative pressure to a fluid-receiving passageway. A connector fluidly connects the drainage lumen to the fluid-receiving passageway. The connector includes an actuation device that, when actuated, provides communication from the pre-evacuated lumen to the fluid-receiving passageway for introducing the negative pressure to the fluid-receiving passageway.

### TECHNICAL FIELD

The present specification generally relates to drainage systems and methods for draining bodily fluid and, more specifically, to drainage systems and methods including a multi-lumen conduit with a pre-evacuated lumen for introducing a negative pressure for draining and collecting bodily fluids.

### BACKGROUND

Various drainage systems are known for a variety of conditions. As an example, percutaneous drainage devices are known for draining cysts, abscesses and biliary drainage. Also, there are a number of longer-term drainage systems for managing recurrent pleural effusions and ascites at home. Two such systems are the PleurX™ and PeritX™ drainage systems, commercially available from Becton, Dickinson and Company. These systems utilize a catheter and drainage bottles that collect fluid. The catheter is inserted, typically as a simple outpatient procedure, in the chest for draining pleural effusions or in the abdomen for ascites. An end of the catheter stays outside of the body, hidden under a bandage when not in use. To drain, the end of the catheter is connected to a drainage line on the bottle that is pre-evacuated to provide a negative pressure, and the negative pressure inside the bottle is used to draw out the bodily fluid.

### SUMMARY

According to a first embodiment, a drainage system for draining bodily fluid includes a multi-lumen conduit comprising a drainage lumen configured to receive a bodily fluid therethrough and a pre-evacuated lumen that is pre-evacuated for providing a negative pressure to a fluid-receiving passageway. A connector fluidly connects the drainage lumen to the fluid-receiving passageway. The connector includes an actuation device that, when actuated, provides communication from the pre-evacuated lumen to the fluid-receiving passageway for introducing the negative pressure to the fluid-receiving passageway.

According to another embodiment, a method of forming a drainage system for draining a bodily fluid from a body cavity is provided. The method includes connecting a first connector to a proximal end of a multi-lumen conduit. The multi-lumen conduit includes a drainage lumen configured to receive a bodily fluid therethrough and a pre-evacuated lumen that is pre-evacuated for providing a negative pressure to a fluid-receiving passageway. A second connector is connected to a distal end of the multi-lumen conduit. The first connector and second connector configured to connect the drainage lumen to the fluid-receiving passageway. One of the first connector and second connector includes an actuation device that, when actuated, provides communication from the pre-evacuated lumen to the fluid-receiving passageway for introducing the negative pressure to the fluid-receiving passageway.

According to another embodiment, a multi-lumen conduit device includes a drainage lumen configured to receive a bodily fluid therethrough and a pre-evacuated lumen that is pre-evacuated for providing a negative pressure to a fluid-receiving passageway. A connector fluidly connects the drainage lumen to the fluid-receiving passageway. The connector includes an actuation device that, when actuated, provides communication from the pre-evacuated lumen to the fluid-receiving passageway for introducing the negative pressure to the fluid-receiving passageway.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a drainage system including a multi-lumen drainage conduit, according to one or more embodiments shown and described herein;

FIG. 2 schematically depicts a side view of a multi-lumen conduit drainage device including the multi-lumen drainage conduit of FIG. 1;

FIG. 3 schematically illustrates a section side view of the multi-lumen conduit drainage device of FIG. 2, according to one or more embodiments shown and described herein;

FIG. 4 schematically illustrates an exploded view of the multi-lumen conduit drainage device of FIG. 2, according to one or more embodiments shown and described herein;

FIG. 5 schematically illustrates a section view of a pre-evacuated lumen for use with a multi-lumen drainage conduit, according to one or more embodiments shown and described herein; and

FIG. 6 schematically illustrates a section view of another pre-evacuated lumen for use with a multi-lumen drainage conduit, according to one or more embodiments shown and described herein.

### DETAILED DESCRIPTION

Embodiments described herein are generally directed to drainage systems for draining bodily fluids. The drainage system includes a multi-lumen conduit that includes a drainage lumen configured to receive a bodily fluid therethrough and a pre-evacuated lumen that is pre-evacuated for providing a negative pressure to a fluid-receiving passageway. A connector fluidly connects the drainage lumen to the fluid-receiving passageway. The connector includes an actuation device that, when actuated, provides communication from the pre-evacuated lumen to the fluid-receiving passageway for introducing the negative pressure to the fluid-receiving passageway.

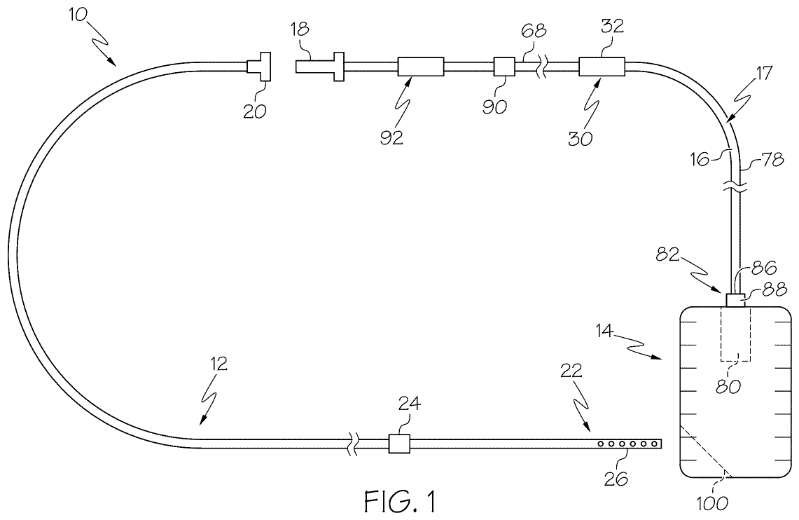

Referring to FIG. 1, a drainage system 10 for use in draining bodily fluids from body cavities includes a drainage catheter 12 that may be partially implanted in a patient's body and a fluid-receiving container 14 that can be fluidly connected to the drainage catheter 12. The fluid-receiving container 14 may be connected to the drainage catheter 12 by a drainage line 16 that extends between the fluid-receiving container 14 and the drainage catheter 12 providing a fluid-receiving passageway 17 therebetween. As used herein, the terms “catheter,” “line,” “conduit” are meant to be used interchangeably. In some embodiments, the drainage line 16 may include a valve connector 18 that connects to a valve 20. As an example, the valve 20 may be a two-piece valve that is normally closed, but that opens with insertion of the valve connector 18 to allow bodily fluid to flow through the drainage line 16. A distal end portion 22 of the drainage catheter 12 may be implanted in the patient's body for pleural or peritoneal drainage or other body location for draining a bodily fluid. In some embodiments, the distal end portion 22 may include a sealing cuff 24 and fenestrations 26 through which the bodily fluid may enter the drainage catheter 12 and be carried from the pleural, peritoneal or other cavity space.

A multi-lumen drainage conduit 30 is provided along the drainage line 16. The multi-lumen drainage conduit 30 may include a single outer wall 32 providing a smooth, continuous outer perimeter and may be formed of a single tube having multiple lumens, as will be described in greater detail below. In other embodiments, the multi-lumen drainage conduit 30 may be formed of separately formed tubes that are connected together by any suitable method, such as welding, adhesives, etc.

The multi-lumen drainage conduit 30 includes a pre-evacuated lumen that is used to supply negative pressure through the drainage conduit 16 and the drainage catheter 12. The negative pressure provides a suction to automatically draw bodily fluid from the cavity in which the drainage catheter 12 is inserted toward the fluid-receiving container 14. As used herein, the terms “negative pressure,” “suction,” vacuum” and “partial vacuum” may be used interchangeably and refer to a force over an area produced by a difference in pressure. Pressure may be referred to as “negative” in reference to atmospheric pressure (i.e., gauge pressure). It should be noted that while using the multi-lumen drainage conduit 30 for draining bodily fluids from body cavities is described primarily herein, the multi-lumen drainage conduit 30 may be used to drain bodily fluids from other locations, such as wounds.

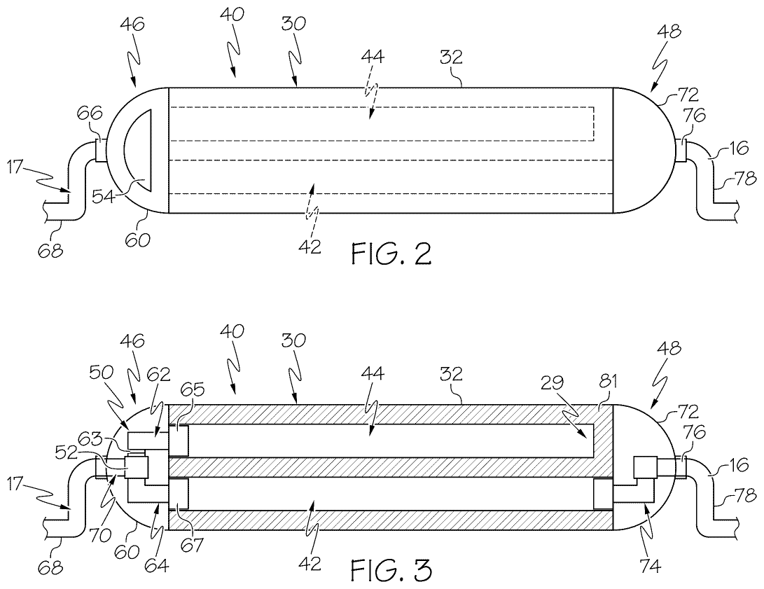

FIGS. 2 and 3 illustrate a multi-lumen conduit drainage device 40 in isolation that includes the multi-lumen drainage conduit 30. As can be best seen in the schematic section view of FIG. 3, the multi-lumen drainage conduit 30 includes a drainage lumen 42 and may extend alongside the pre-evacuated lumen 44. The multi-lumen drainage device 40 further includes a proximal end connector 46 and a distal end connector 48. As used herein, the terms “proximal” and “distal”, in this usage, refer respectively to a user-end (patient-end) of the device or related object and a drainage-end of the device or related object.

The proximal end connector 46 fluidly connects the pre-evacuated lumen 44 to the fluid-receiving passageway 17. For example, as shown in FIG. 3, the proximal end connector 46 may include a connecting passageway 50 that is originally closed, for example, using an actuatable release valve 52 and that is opened using an actuation member 54 (FIG. 2) by manually actuating the actuation member 54 when it is desired to introduce negative pressure to the fluid-receiving passageway 17. In the illustrated example, the valve 52 is a button valve that opens when a button (also represented by element 54) is depressed. The valve 52 may stay open after depressing and releasing the button 54, or the valve may be normally closed and biased toward the closed position by a biasing element such as a spring. In the normally closed embodiment, the valve 52 returns to its closed position once the button 54 is released. While button 54 is shown, any suitable actuation members may be used, such as levers, knobs, dials, slides, etc. As another example, an actuation member may be used to break a seal closing the connecting passageway 50. In some embodiments, the actuation device and connector may be separate components. For example, two connectors may be used, one with an actuation device and fluidly connected to the pre-evacuated lumen and another connector without an actuation device and fluidly connected to the drainage lumen 42. In some embodiments, the drainage lumen 42 may always be open to the fluid-receiving passageway 17 or may be closed and then open.

The connecting passageway 50 is provided within a housing 60 and includes a negative pressure leg 62 that extends to the pre-evacuated lumen 44 and a drainage leg 64 that extends to the drainage lumen 42. The negative pressure and drainage legs 62 and 64 may be sealingly connected to the pre-evacuated and drainage lumens 44 and 42 by connecting stubs 65 and 67. A tube connecting stub 66 may be sealingly connected to a proximal portion of the fluid-receiving passageway 17, namely a first tube 68 of the drainage line 16 to provide fluid communication between the connecting passageway 50 and the fluid-receiving passageway 17. In some embodiments, the valve 52 may be located at the junction between a fluid inlet leg 70 and the negative and drainage legs 62 and 64. The valve 52 may be a 3-way valve that controls fluid communication from the fluid inlet leg 70 to both the negative and drainage legs 62 and 64. In some embodiments, the valve 52 may only release and prevent negative pressure from being released from the pre-evacuated lumen 44 to the fluid-receiving passageway 17 and always allow the flow of bodily fluid into the drainage lumen 42 and then to pass to the fluid-receiving container 14. In some embodiments, a hydrophobic or hydrophilic filter 63 may be located between the connector 46 and the pre-evacuated lumen 44 that inhibits liquid from entering the pre-evacuated lumen 44 once the filter 63 is wetted. Such a filter arrangement can cause substantially all of the bodily fluid to bypass the pre-evacuated lumen 44 and be collected in the fluid-receiving container 14 via the drainage lumen 42. In other embodiments, a seal (also represented by element 52) may be used and the actuation member 54 may be used to pierce the seal 52 thereby releasing the negative pressure into the fluid-receiving passageway 17.

The distal end connector 48 may or may not include all the features of the proximal end connector 46. In some embodiments, the connector 48 at the distal end may be replaced by the connector 46 at the proximal end and vice versa. For example, in the illustrated embodiment, the distal end connector 48 includes a housing 72 and a connecting passageway 74. The connecting passageway 74 may only fluidly connect the drainage lumen 42 to the drainage line 16 and not connect the pre-evacuated lumen 44 to the fluid-receiving passageway 17 like proximal end connector 46. A tube connecting stub 76 may be sealingly connected to a distal portion of the fluid-receiving passageway, namely a second tube 78 of the drainage line 16 to provide fluid communication between the connecting passageway 74 and the fluid-receiving passageway 17. A connecting stub 77 may sealingly connect the connecting passageway 74 to the drainage lumen 42. In some embodiments, a distal end 79 of the pre-evacuated lumen 44 may be closed by a seal 81. As examples, the seal 81 may be formed by the outer wall 32 or applied to the outer wall 32, covering the pre-evacuated lumen 44. As another example, the distal end connector 48 may seal the pre-evacuated lumen 44.

Referring again to FIG. 1, a one-way valve 80 may be located along the drainage line 17 downstream of the multi-lumen drainage conduit 30. The terms “downstream” and “upstream” are with reference to drainage flow during a drainage operation with a downstream direction being toward the fluid-receiving container 14 and upstream direction being away from the fluid-receiving container 14. In the illustrated example, the one-way valve 80 is a flutter valve that is located at an inlet 82 to a fluid-receiving volume 84 of the fluid-receiving container 14. An end 86 of the second tube 78 may be fluidly connected to the one-way valve 80 at the inlet 82. In some embodiments, the end 86 may be connected to a stub tube 88. While the one-way valve 80 is located at the inlet 82, a one-way valve may be located anywhere along the second tube 78 between the fluid-receiving volume 84 and the connecting multi-lumen drainage conduit 30. For example, it may be desirable to provide the one-way valve 80 closer to the multi-lumen drainage conduit 30 to reduce a distance between the connector 18 and the one-way valve 80 thereby reducing an air-filled volume in the drainage line 16 when providing negative pressure. In some embodiments, the one-way valve 80 may be located in the drainage lumen 42.

Another one-way valve 90 may be located along the drainage line 16 upstream of the multi-lumen drainage conduit 30. The one-way valve 90 may be located anywhere along the drainage line 16 between the multi-lumen drainage conduit 30 and the connector 18. During a drainage operation, the one-way valve 90 prevents the flow of air and liquid upstream toward the body cavity. Further, a flow control device 92, such as a tubing clamp, may be used to constrict or release the drainage line 16, which can be used to decrease or increase fluid flow rate as desired by the user.

In operation, once the connector 18 is fluidly connected to the drainage catheter 12 through the valve 20, a drainage operation may be initiated with the drainage catheter 12 already implanted within a body cavity. To initiate the drainage operation, the fluid-receiving container 14 (e.g., a bag) may be placed on a floor such that it is lower than the body cavity. Depending on locations and orientations of the body cavity, the drainage conduit 16, the fluid-receiving container 14 and a pressure difference between the body cavity and outside atmosphere, drainage flow may begin without use of the negative pressure within the pre-evacuated lumen 44. This is considered gravity flow.

In some instances, it may be desirable to introduce a lower pressure within the fluid-receiving passageway 17 of the drainage catheter 12 to initiate or increase bodily fluid flow. To this end, the actuation member 54 of the proximal end connector 46 is actuated thereby opening the valve 52. A suction is communicated through the connecting passageway 50 and to the fluid-receiving passageway 17. The other one-way valve 90 allows the negative pressure to be communicated therethrough to the drainage catheter 12. The flow control device 35 may be used to constrict or release the drainage line 16, which can be used to decrease or increase fluid flow rate as desired by the user. The fluid-receiving container 14 may be formed of a plastic sheet material. In some embodiments, the plastic sheet material includes a line of weakness 100 that facilitates tearing of the fluid-receiving container 14 and pouring out of the contents.

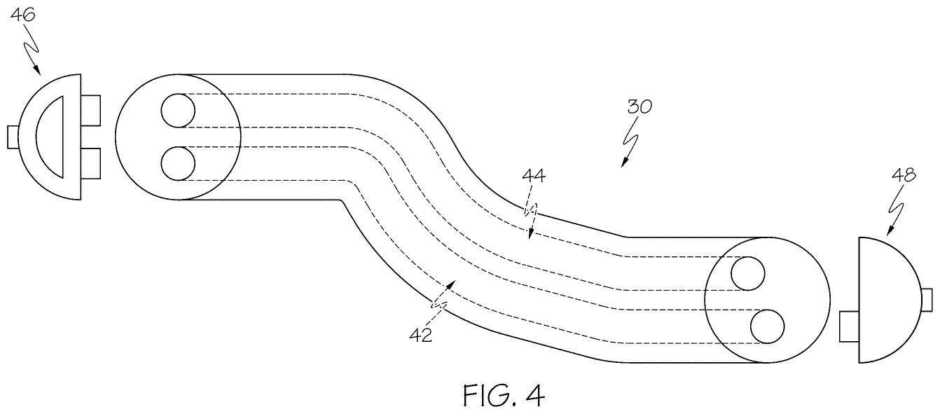

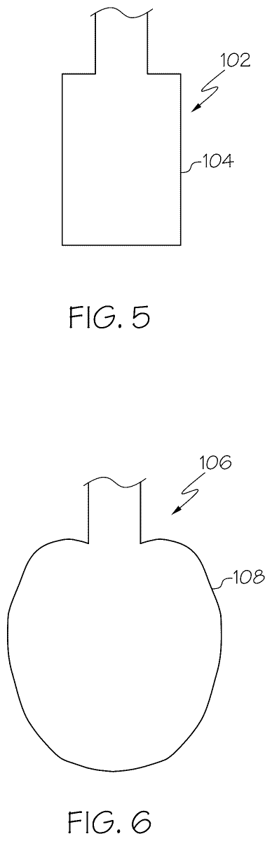

Referring to FIG. 4, the multi-lumen drainage conduit 30 may be formed of any suitable materials including relatively soft and relatively hard materials. For example, the multi-lumen conduit 30 may be formed of polyvinyl chloride (PVC), rubber, silicon, etc. or combinations thereof. The multi-lumen conduit 30 may be formed by any suitable method, such as extrusion, casting, molding, etc. or combinations of methods. Further, while the drainage lumen 42 and the pre-evacuated lumen 44 are illustrated as being straight and parallel, they may be any suitable shape and may not be the same shape. As examples, FIG. 5 illustrates an embodiment of a pre-evacuated lumen 102 having a stepped-out portion 104 of increased inner diameter, adding additional volume to the pre-evacuated lumen 102. FIG. 6 illustrates another example pre-evacuated lumen 106 having a rounded portion 108, also adding additional volume to the pre-evacuated lumen 106.

The above-described multi-lumen conduit drainage devices can be provided for drainage systems and include a multi-lumen drainage conduit with a pre-evacuated lumen and a connector that acts as a release device that can be used to release a negative pressure within the pre-evacuated lumen into a fluid-receiving passageway that extends from a fluid-receiving container to a body cavity. The connector may include an actuator member, such as a button, lever, etc. that can be actuated (i.e., moved) in order to open a connecting passageway that extends between the pre-evacuated lumen and the fluid-receiving passageway. Such an actuator member arrangement can make it easier for a user to activate than devices that require a higher degree of movement, such as a manual pump, for example.

Embodiments can be described with reference to the following numbered clauses, with certain features laid out in the dependent clauses:

Clause 1: A drainage system for draining bodily fluid, the drainage system comprising:
a multi-lumen conduit comprising a drainage lumen configured to receive a bodily fluid therethrough and a pre-evacuated lumen that is pre-evacuated for providing a negative pressure to a fluid-receiving passageway; and a connector configured to fluidly connect the drainage lumen to the fluid-receiving passageway, the connector comprising an actuation device that, when actuated, provides communication from the pre-evacuated lumen to the fluid-receiving passageway for introducing the negative pressure to the fluid-receiving passageway.

Clause 2: The drainage system of clause 1, wherein the connector is a first connector located at a proximal end of the multi-lumen conduit for connecting to a proximal portion of the fluid-receiving passageway, the drainage system further comprising a second connector located at a distal end of the multi-lumen conduit configured to fluidly connect the drainage lumen to a distal portion of the fluid-receiving passageway so that the drainage lumen forms a fluid path between the proximal and distal portions of the fluid-receiving passageway.

Clause 3: The drainage system of clause 2 further comprising a fluid-receiving body having a fluid-receiving chamber located therein that is configured to receive a bodily fluid from the fluid-receiving passageway.

Clause 4: The drainage system of clause 3 further comprising: a first drainage conduit fluidly connected to the first connector; and a second drainage conduit fluidly connected to the second connector and the fluid-receiving body, the first and second drainage conduits providing at least portions of the fluid-receiving passageway.

Clause 5: The drainage system of any of clauses 1-4 further comprising a hydrophobic or hydrophilic filter in the connector that inhibits liquid from entering the pre-evacuated lumen from the fluid-receiving passageway.

Clause 6: The drainage system of any of clauses 1-5, wherein the connector comprises a release valve connected between the pre-evacuated lumen and the fluid-receiving passageway, and the actuation device opens the release valve upon actuation.

Clause 7: The actuation device of any of clauses 1-6, wherein the connector comprises a seal preventing fluid communication between the pre-evacuated lumen and the fluid-receiving passageway and the actuation device pierces the seal upon actuation.

Clause 8: The actuation device of any of clauses 1-7, wherein the actuation device comprises a button or lever.

Clause 9: A method of forming a drainage system for draining a bodily fluid from a body cavity, the method comprising: connecting a first connector to a proximal end of a multi-lumen conduit, the multi-lumen conduit comprising a drainage lumen configured to receive a bodily fluid therethrough and a pre-evacuated lumen that is pre-evacuated for providing a negative pressure to a fluid-receiving passageway; and connecting a second connector to a distal end of the multi-lumen conduit, the first connector and second connector configured to connect the drainage lumen between a proximal portion and a distal portion of a fluid-receiving passageway; wherein one of the first connector and second connector comprises an actuation device that, when actuated, provides communication from the pre-evacuated lumen to the fluid-receiving passageway for introducing the negative pressure to the fluid-receiving passageway.

Clause 10: The method of clause 9 further comprising fluidly connecting the fluid-receiving passageway to a fluid-receiving body having a fluid-receiving chamber located therein that is configured to receive a bodily fluid from the fluid-receiving passageway.

Clause 11: The method of claim 10 further comprising: connecting a first drainage conduit to the first connector; and connecting a second drainage conduit to the second connector and the fluid-receiving body, the first and second drainage conduits providing at least portions of the fluid-receiving passageway.

Clause 12: The method of any of clauses 9-11 further comprising placing a hydrophobic or hydrophilic filter in the one of the first connector that inhibits liquid from entering the pre-evacuated lumen.

Clause 13: The method of any of clauses 9-12, wherein the connector comprises a release valve connected between the pre-evacuated lumen and the fluid-receiving passageway, and the actuation device opens the release valve upon actuation.

Clause 14: The method of any of clauses 9-13, wherein the connector comprises a seal preventing fluid communication between the pre-evacuated lumen and the fluid-receiving passageway and the actuation device pierces the seal upon actuation.

Clause 15: A multi-lumen conduit device comprising: a drainage lumen configured to receive a bodily fluid therethrough and a pre-evacuated lumen that is pre-evacuated for providing a negative pressure to a fluid-receiving passageway; and a connector configured to fluidly connect the drainage lumen to the fluid-receiving passageway, the connector comprising an actuation device that, when actuated, provides communication from the pre-evacuated lumen to the fluid-receiving passageway for introducing the negative pressure to the fluid-receiving passageway.

Clause 16: The multi-lumen conduit device of clause 15, wherein the connector is a first connector located at a proximal end of the multi-lumen conduit for connecting to a proximal portion of the fluid-receiving passageway, the drainage system further comprising a second connector located at a distal end of the multi-lumen conduit configured to fluidly connect the drainage lumen to a distal portion of the fluid-receiving passageway so that the drainage lumen forms a fluid path between the proximal and distal portions of the fluid-receiving passageway.

Clause 17: The multi-lumen conduit device of claim 15 or 16 further comprising a hydrophobic or hydrophilic filter in the connector that inhibits liquid from entering the pre-evacuated lumen from the fluid-receiving passageway.

Clause 18: The multi-lumen conduit device of any of claims 15-17, wherein the connector comprises a release valve connected between the pre-evacuated lumen and the fluid-receiving passageway, and the actuation device opens the release valve upon actuation.

Clause 19: The multi-lumen conduit device of any of claims 15-18, wherein the connector comprises a seal preventing fluid communication between the pre-evacuated lumen and the fluid-receiving passageway and the actuation device pierces the seal upon actuation.

Clause 20: The multi-lumen conduit device of and of clauses 15-19, wherein the actuation device comprises a button or lever.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A drainage system for draining bodily fluid, the drainage system comprising:
a multi-lumen conduit comprising a drainage lumen configured to receive a bodily fluid therethrough and a pre-evacuated lumen that is pre-evacuated for providing a negative pressure to a fluid-receiving passageway; and
a connector configured to fluidly connect the drainage lumen to the fluid-receiving passageway, the connector comprising an actuation device that, when actuated, provides communication from the pre-evacuated lumen to the fluid-receiving passageway for introducing the negative pressure to the fluid-receiving passageway.

2. The drainage system of claim 1, wherein the connector is a first connector located at a proximal end of the multi-lumen conduit for connecting to a proximal portion of the fluid-receiving passageway, the drainage system further comprising a second connector located at a distal end of the multi-lumen conduit configured to fluidly connect the drainage lumen to a distal portion of the fluid-receiving passageway so that the drainage lumen forms a fluid path between the proximal and distal portions of the fluid-receiving passageway.

3. The drainage system of claim 2 further comprising a fluid-receiving container having a fluid-receiving chamber located therein that is configured to receive a bodily fluid from the fluid-receiving passageway.

4. The drainage system of claim 3 further comprising:
a first drainage tube fluidly connected to the first connector; and
a second drainage tube fluidly connected to the second connector and the fluid-receiving container, the first and second drainage tubes providing at least portions of the fluid-receiving passageway.

5. The drainage system of claim 1 further comprising a hydrophobic or hydrophilic filter in the connector that inhibits liquid from entering the pre-evacuated lumen from the fluid-receiving passageway.

6. The drainage system of claim 1, wherein the connector comprises a release valve connected between the pre-evacuated lumen and the fluid-receiving passageway, and the actuation device opens the release valve upon actuation.

7. The drainage device of claim 1, wherein the connector comprises a seal preventing fluid communication between the pre-evacuated lumen and the fluid-receiving passageway and the actuation device pierces the seal upon actuation.

8. The drainage device of claim 1, wherein the actuation device comprises a button or lever.

9. A method of forming a drainage system for draining a bodily fluid from a body cavity, the method comprising:
connecting a first connector to a proximal end of a multi-lumen conduit, the multi-lumen conduit comprising a drainage lumen configured to receive a bodily fluid therethrough and a pre-evacuated lumen that is pre-evacuated for providing a negative pressure to a fluid-receiving passageway; and
connecting a second connector to a distal end of the multi-lumen conduit, the first connector and second connector configured to connect the drainage lumen between a proximal portion and a distal portion of the fluid-receiving passageway;
wherein one of the first connector and second connector comprises an actuation device that, when actuated, provides communication from the pre-evacuated lumen to the fluid-receiving passageway for introducing the negative pressure to the fluid-receiving passageway.

10. The method of claim 9 further comprising fluidly connecting the fluid-receiving passageway to a fluid-receiving container having a fluid-receiving chamber located therein that is configured to receive a bodily fluid from the fluid-receiving passageway.

11. The method of claim 10 further comprising:
connecting a first drainage tube to the first connector; and
connecting a second drainage tube to the second connector and the fluid-receiving container, the first and second drainage tubes providing at least portions of the fluid-receiving passageway.

12. The method of claim 9 further comprising placing a hydrophobic or hydrophilic filter in the one of the first connector and the second connector that inhibits liquid from entering the pre-evacuated lumen.

13. The method of claim 9, wherein the connector comprises a release valve connected between the pre-evacuated lumen and the fluid-receiving passageway, and the actuation device opens the release valve upon actuation.

14. The method of claim 9, wherein the connector comprises a seal preventing fluid communication between the pre-evacuated lumen and the fluid-receiving passageway and the actuation device pierces the seal upon actuation.

15. A multi-lumen conduit device comprising:
a drainage lumen configured to receive a bodily fluid therethrough and a pre-evacuated lumen that is pre-evacuated for providing a negative pressure to a fluid-receiving passageway; and
a connector that fluidly connects the drainage lumen to the fluid-receiving passageway, the connector comprising an actuation device that, when actuated, provides communication from the pre-evacuated lumen to the fluid-receiving passageway for introducing the negative pressure to the fluid-receiving passageway.

16. The multi-lumen conduit device of claim 15, wherein the connector is a first connector located at a proximal end of the multi-lumen conduit for connecting to a proximal portion of the fluid-receiving passageway, the drainage system further comprising a second connector located at a distal end of the multi-lumen conduit configured to fluidly connect the drainage lumen to a distal portion of the fluid-receiving passageway so that the drainage lumen forms a fluid path between the proximal and distal portions of the fluid-receiving passageway.

17. The multi-lumen conduit device of claim 15 further comprising a hydrophobic or hydrophilic filter in the connector that inhibits liquid from entering the pre-evacuated lumen.

18. The multi-lumen conduit device of claim 15, wherein the actuation device opens a release valve upon actuation.

19. The multi-lumen conduit device of claim 15, wherein the connector comprises a release valve connected between the pre-evacuated lumen and the fluid-receiving passageway, and the actuation device opens the release valve upon actuation.

20. The multi-lumen conduit device of claim 15, wherein the connector comprises a seal preventing fluid communication between the pre-evacuated lumen and the fluid-receiving passageway and the actuation device pierces the seal upon actuation.
